SEQUENCE LISTING (35 sequences)

SEQ ID NO: 1 - DNA, 15 bp, synthetic construct
  source 1..15: mol_type = other DNA
agcagaggaggagaa

SEQ ID NO: 2 - DNA, 20 bp, synthetic construct
  source 1..20: mol_type = other DNA
ttgttcagaagtatcctttc

SEQ ID NO: 3 - DNA, 22 bp, synthetic construct
  source 1..22: mol_type = other DNA
agaaagaaggaagaggaggaga

SEQ ID NO: 4 - DNA, 10 bp, synthetic construct
  source 1..10: mol_type = other DNA
taatccggtt

SEQ ID NO: 5 - DNA, 10 bp, synthetic construct
  source 1..10: mol_type = other DNA
attaggccaa

SEQ ID NO: 6 - DNA, 10 bp, synthetic construct
  source 1..10: mol_type = other DNA
attcggccaa

SEQ ID NO: 7 - DNA, 10 bp, synthetic construct
  source 1..10: mol_type = other DNA
attagaccaa

SEQ ID NO: 8 - DNA, 25 bp, synthetic construct
  source 1..25: mol_type = other DNA
agcagaggaggagaatgaagaaaga

SEQ ID NO: 9 - DNA, 25 bp, synthetic construct
  source 1..25: mol_type = other DNA
tctttcttcattctcctcctctgct

SEQ ID NO: 10 - DNA, 69 bp, synthetic construct
  source 1..69: mol_type = other DNA
gaattccaagcttcatgctacgttggagaaggaggagaaggaaagagtcctctatacgagatctggatc

SEQ ID NO: 11 - DNA, 69 bp, synthetic construct
  source 1..69: mol_type = other DNA
gatccagatctcgtatagaggactctttccttctcctccttctccaacgtagcatgaagcttggaattc

SEQ ID NO: 12 - DNA, 69 bp, synthetic construct
  source 1..69: mol_type = other DNA
gaattccaagcttcatgctacgttggagaagtaggagaaggaaagagtcctctatacgagatctggatc

SEQ ID NO: 13 - DNA, 69 bp, synthetic construct
  source 1..69: mol_type = other DNA
gatccagatctcgtatagaggactctttccttctcctacttctccaacgtagcatgaagcttggaattc

SEQ ID NO: 14 - DNA, 69 bp, synthetic construct
  source 1..69: mol_type = other DNA
gaattccaagcttcatgctacgttggagaagttggagaaggaaagagtcctctatacgagatctggatc

SEQ ID NO: 15 - DNA, 69 bp, synthetic construct
  source 1..69: mol_type = other DNA
gatccagatctcgtatagaggactctttccttctccaacttctccaacgtagcatgaagcttggaattc

SEQ ID NO: 16 - DNA, 69 bp, synthetic construct
  source 1..69: mol_type = other DNA
gaattccaagcttcatgctacgttggagaagtaggagaagtaaagagtcctctatacgagatctggatc

SEQ ID NO: 17 - DNA, 69 bp, synthetic construct
  source 1..69: mol_type = other DNA
gatccagatctcgtatagaggactctttacttctcctacttctccaacgtagcatgaagcttggaattc

SEQ ID NO: 18 - DNA, 69 bp, synthetic construct
  source 1..69: mol_type = other DNA
gaattccaagcttcatgctacgttggagaagcaggagaaggaaagagtcctctatacgagatctggatc

SEQ ID NO: 19 - DNA, 69 bp, synthetic construct
  source 1..69: mol_type = other DNA
gatccagatctcgtatagaggactctttccttctcctgcttctccaacgtagcatgaagcttggaattc

SEQ ID NO: 20 - DNA, 23 bp, synthetic construct
  source 1..23: mol_type = other DNA
gagaaaggaagaggaggaagagg

SEQ ID NO: 21 - DNA, 23 bp, synthetic construct
  source 1..23: mol_type = other DNA
gagaaaggaagaggaagaagagg

SEQ ID NO: 22 - DNA, 23 bp, synthetic construct
  source 1..23: mol_type = other DNA
gagaaaggaagaggacgaagagg

SEQ ID NO: 23 - DNA, 23 bp, synthetic construct
  source 1..23: mol_type = other DNA
gagaaaggaagaggatgaagagg

SEQ ID NO: 24 - DNA, 20 bp, synthetic construct
  source 1..20: mol_type = other DNA
aaaggaagaggaggaagagg

SEQ ID NO: 25 - DNA, 17 bp, synthetic construct
  source 1..17: mol_type = other DNA
ggaagaggaggaagagg

SEQ ID NO: 26 - DNA, 14 bp, synthetic construct
  source 1..14: mol_type = other DNA
agaggaggaagagg

SEQ ID NO: 27 - DNA, 10 bp, synthetic construct
  source 1..10: mol_type = other DNA
ttggcctaat

SEQ ID NO: 28 - DNA, 10 bp, synthetic construct
  source 1..10: mol_type = other DNA
ttggactaat

SEQ ID NO: 29 - DNA, 10 bp, synthetic construct
  source 1..10: mol_type = other DNA
tcggcctaat

SEQ ID NO: 30 - DNA, 90 bp, synthetic construct
  source 1..90: mol_type = other DNA
aaacccccactgcaatgattatcgcaatgggggaaagaggggacttaaagcatatgtttagctttgaatacttaaaattctcttgctatt

SEQ ID NO: 31 - DNA, 90 bp, synthetic construct
  source 1..90: mol_type = other DNA
aatagcaagagaattttaagtattcaaagctaaacatatgctttaagtcccctctttcccccattgcgataatcattgcagtgggggttt

SEQ ID NO: 32 - DNA, 18 bp, synthetic construct
  source 1..18: mol_type = other DNA
aggggagaaagggggtaa

SEQ ID NO: 33 - DNA, 16 bp, synthetic construct
  source 1..16: mol_type = other DNA
gggagaaagggggtaa

SEQ ID NO: 34 - DNA, 14 bp, synthetic construct
  source 1..14: mol_type = other DNA
gagaaagggggtaa

SEQ ID NO: 35 - DNA, 12 bp, synthetic construct
  source 1..12: mol_type = other DNA
gaaagggggtaa

(19) United States
(12) Patent Application Publication
(10) Pub. No.: US 2025/0043277 A1
(43) Pub. Date: Feb. 6, 2025

(54) TRIPLE-STRANDED NUCLEIC ACID COMPRISING TFO, KITS AND METHODS FOR DETECTING TARGET SEQUENCE BY USING TFO

(71) Applicant: Chang Gung University, Taoyuan City (TW)

(72) Inventors: Chiuan-Chian CHIOU, Taoyuan City (TW); Ya-Han CHANG, Taoyuan City (TW); Wei CHANG, Taoyuan City (TW)

(21) Appl. No.: 18/784,048

(22) Filed: Jul. 25, 2024

(30) Foreign Application Priority Data
Aug. 1, 2023 (TW) 112128927

(51) Int. Cl. C12N 15/11; C12Q 1/6806; C12Q 1/6869

(52) U.S. Cl. CPC C12N 15/11; C12Q 1/6806; C12Q 1/6869; C12N 2310/15; C12N 2310/153; C12N 2310/335

(57) ABSTRACT

Disclosed herein is a triple-stranded nucleic acid comprising a double-stranded nucleic acid and a triplex forming oligonucleotide (TFO), in which the double-stranded nucleic acid comprises a first strand and a second strand complementary to the first strand, and the TFO binds to the first strand. According to some embodiments of the present disclosure, the second strand comprises a plurality of modified nucleotides independently selected from the group consisting of 5-fluoro-uridine, 5-chloro-uridine, 5-bromo-uridine and 5-formyl-uridine nucleotides. Also disclosed herein are kits and methods of detecting a target sequence in a double-stranded nucleic acid.

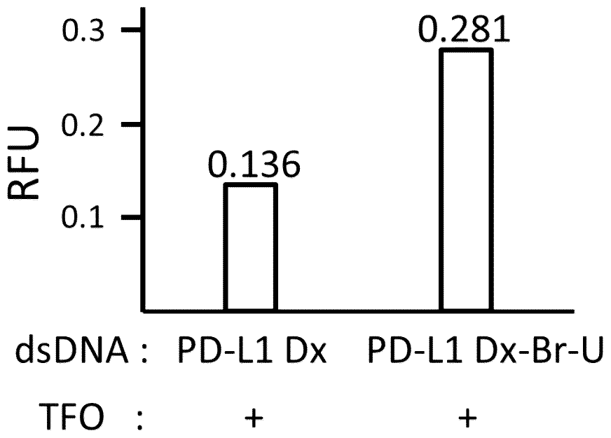

### CROSS-REFERENCE TO RELATED APPLICATIONS

This application relates to and claims the benefit of TW Application No. 112128927, filed Aug. 1, 2023; the content of the application is incorporated herein by reference in its entirety.

### SEQUENCE LISTING XML

The present application is being filed along with a Sequence Listing XML in electronic format. The Sequence Listing XML is provided as a XML file entitled P4146US SEQ_AF, created Jun. 17 2024, which is 32 Kb in size. The information in the electronic format of the Sequence Listing XML is incorporated herein by reference in its entirety.

### BACKGROUND OF THE INVENTION

### 1. Field of the Invention

The present disclosure in general relates to the field of sequence detection. More particularly, the present disclosure relates to methods of detecting a target sequence in a double-stranded nucleic acid by using primer pair and triplex forming oligonucleotide (TFO).

### 2. Description of Related Art

TFO is a single-stranded deoxyribonucleic acid (ssDNA) that may bind to the major groove of double-stranded DNA (dsDNA) in a sequence-specific manner and forms a triple helix. Based on the nucleotide sequence, TFO may be classified as three groups, including, (i) the TC-rich TFO, in which the TFO is composed of thymine (T) and cytosine (C) nucleotides that forms parallel bonds with respect to the polypurine strand of dsDNA, i.e., Hoogsteen base pairing (such as T*A=T, or C*G≡C, where the symbols “=” and “≡” denote Watson-Crick base pairing between two strands of dsDNA, and the symbol “*” denotes Hoogsteen base pairing between the TFO and the polypurine strand of dsDNA); (ii) GA-rich TFO, in which the TFO is composed of guanine (G) and adenine (A) nucleotides that forms anti-parallel bonds with respect to the polypurine strand of dsDNA, i.e., reverse Hoogsteen base pairing (such as G*G≡C or A*A=T); and (iii) GT-rich TFO, in which the TFO is composed of guanine (G) and thymine (T) nucleotides that can adopt both parallel and anti-parallel binding configuration, i.e., G*G≡C and T*A=T.

Based on the sequence specificity, TFO has been widely used in various studies, including transcription regulation, genome modification (e.g., site-specific mutagenesis, site-specific recombination, site-specific slicing and gene silencing), DNA repair, and in situ hybridization. Also, TFO may serve as a therapeutic agent for treating diseases via altering (e.g., enhancing or inhibiting) specific gene expression; or as a diagnostic agent (e.g., a probe) for detecting the expression or mutation of disease-associated genes. However, the triplex formation is restricted to a DNA region with purines on one strand and pyrimidines on the other strand, in which TFO can only bind to the purine-rich strand. It is reported that addition of one pyrimidine nucleotide in the purine-rich strand would lead to about 30-fold decrease in TFO binding affinity. For the purpose of improving the binding affinity of TFO to pyrimidine-containing strand, a great effort has been made to modify the sequence or structure of TFO. Unfortunately, the modification may adversely alter the stability and/or solubility of TFO, and thus, none of the modification provides a satisfactory result.

In view of the forging, there exists in the related art a need for a novel method for improving the binding affinity of TFO to pyrimidine nucleotide of dsDNA.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a triple-stranded nucleic acid comprising a double-stranded nucleic acid and a TFO, wherein the double-stranded nucleic acid is a double-stranded DNA comprising a first strand and a second strand complementary to the first strand. According to some embodiments of the present disclosure, the first strand of the double-stranded nucleic acid is a polypurine sequence, and the second strand of the double-stranded nucleic acid is a polypyrimidine sequence, wherein the first strand binds to the TFO via Hoogsteen base pairing or reverse Hoogsteen base pairing, and the second strand comprises a plurality of modified nucleotides. In the embodiments, the modified nucleotides are independently selected from the group consisting of 5-fluoro-uridine, 5-chloro-uridine, 5-bromo-uridine, and 5-formyl-uridine nucleotides.

Preferably, one or less than one pyrimidine nucleotide is present in the polypurine sequence.

According to some embodiments, the double-stranded nucleic acid is produced by a reaction catalyzed by a polymerase (e.g., a DNA polymerase); for example, polymerase chain reaction (PCR), loop-mediated isothermal amplification (LAMP), recombinase polymerase amplification (RPA), etc.

According to certain embodiments, the double-stranded nucleic acid consists of 9-40 base pairs (bps). Preferably, the TFO and the double-stranded nucleic acid are same in length.

The second aspect of the present disclosure is directed to a method of detecting a target sequence in a double-stranded nucleic acid. The method comprises,
(a) mixing the double-stranded nucleic acid with PCR reagents that comprise a forward primer, a reverse primer, a DNA polymerase, a reaction buffer, and deoxynucleotide triphosphates (dNTPs), wherein
   the forward primer is linked to a first fluorophore;
   the dNTPs consist of a deoxyadenosine triphosphate (dATP), a deoxycytidine triphosphate (dCTP), a deoxyguanosine triphosphate (dGTP), and a modified deoxyuridine triphosphate (dUTP), wherein the modified dUTP is selected from the group consisting of 5-bromo-dUTP, 5-chloro-dUTP, 5-fluoro-dUTP, and 5-formyl-dUTP;
(b) subjecting the mixture of step (a) to PCR;
(c) mixing the amplified product of step (b) with a TFO thereby forming a triplex complex, wherein the TFO is linked to a second fluorophore, wherein one of the first and second fluorophores is a donor fluorophore, and the other of the first and second fluorophores is an acceptor fluorophore;
(d) irradiating the product of step (c) with a light having a first wavelength to excite the donor fluorophore; and
(e) measuring the signal emitted from the acceptor fluorophore at a second wavelength to detect the target sequence.

According to the embodiments of the present disclosure, the PCR reagents do not comprise a deoxythymidine triphosphate (dTTP). Accordingly, the thus-obtained PCR product consists of dATP(s), dCTP(s), dGTP(s) and modified dUTP(s). According to certain embodiments, the dUTP is selected from the group consisting of 5-bromo-dUTP (hereinafter as “Br-dUTP”), 5-chloro-dUTP (hereinafter as “Cl-dUTP”), 5-fluoro-dUTP (hereinafter as “F-dUTP”), and 5-formyl-dUTP (hereinafter as “Fml-dUTP”). In one preferred embodiment, the dUTP is the Br-dUTP.

According to some optional embodiments, the method further comprises incubating the product of step (c) at 37° C. to 40° C. for 30-60 minutes thereby forming the triplex complex (i.e., step (c-1)).

Preferably, the PCR reagents further comprise MgCl₂.

According to one exemplary embodiment, the donor fluorophore and acceptor fluorophore are respectively fluorescein isothiocyanate (FITC) and carboxy-X-rhodamine (ROX). In the embodiment, the first and second wavelengths are respectively 485-495 nm and 600-605 nm.

According to some embodiments, the TFO has 9-40 nucleotides in length, and the nucleotide sequence of the TFO is substantially reverse to a strand amplified by the forward primer (i.e., the TFO is substantially reverse complementary to a strand amplified by the reverse primer). Preferably, the TFO has 10-25 nucleotides in length.

According to some embodiments, the first fluorophore is linked to the 5′ end of the forward primer, and the second fluorophore is linked to the 3′ end of the TFO. In one exemplary embodiment, the first fluorophore is the donor fluorophore, and the second fluorophores is the acceptor fluorophore

As could be appreciated, in addition to detecting the target sequence via PCR as described in aforementioned method, the present TFO is also useful in detecting the target sequence via other polymerase-catalyzed methods, for example, LAMP, RPA, etc.

The third aspect of the present disclosure provides a kit for detecting a target sequence in a double-stranded nucleic acid. According to some embodiments, the kit comprises,
a forward primer and a reverse primer configured to amplify the target sequence in PCR, wherein the forward primer is linked to a first fluorophore;
a TFO having 9-40 nucleotides in length, in which the nucleotide sequence of the TFO is substantially reverse to a strand amplified by the forward primer (i.e. the TFO is substantially reverse complementary to a strand amplified by the reverse primer), wherein the TFO is linked to a second fluorophore, wherein one of the first and second fluorophores is a donor fluorophore, and the other of the first and second fluorophores is an acceptor fluorophore; and
dNTPs consisting of a dATP, a dCTP, a dGTP, and a modified dUTP, wherein the modified dUTP is selected from a group consisting of Br-dUTP, Cl-dUTP, F-dUTP, and Fml-dUTP.

The present kit is characterized by not comprising a dTTP.

Preferably, the TFO has 10-25 nucleotides in length.

Preferably, the modified dUTP is the Br-dUTP.

Optionally, the present kit further comprises a DNA polymerase, reaction buffer, and MgCl₂.

In certain embodiments of the present disclosure, the first fluorophore is linked to the 5′ end of the forward primer, and the second fluorophore is linked to the 3′ end of the TFO. In one exemplary embodiment, the first fluorophore is the donor fluorophore, and the second fluorophore is the second fluorophore.

According to some embodiments, one or less than one pyrimidine nucleotide (i.e., T and C nucleotides) is present in the nucleotide sequence of the TFO. Preferably, the TFO consists of purine nucleotides, in which the purine nucleotides may be adenine or guanine nucleotides.

According to one exemplary embodiment, the target sequence is programmed death-ligand 1 (PD-L1). In the embodiment, the forward and reverse primers respectively comprise the nucleotide sequences of SEQ ID NOs: 1 and 2, and the TFO comprises the nucleotide sequence of SEQ ID NO: 3.

Many of the attendant features and advantages of the present disclosure will become better understood with reference to the following detailed description considered in connection with the accompanying drawing.

### BRIEF DESCRIPTION OF THE DRAWING

The present description will be better understood from the following detailed description read in light of the accompanying drawing, where:

The sole FIGURE is a histogram depicting the effect of Br-U modification on binding affinity of dsDNA to TFO according to Example 5 of the present disclosure. PD-L1 Dx: PD-L1 gene fragment without Br-U modification; PDL1 Dx-Br-U: PD-L1 gene fragment with Br-U modification. RFU: relative fluorescence units.

### DETAILED DESCRIPTION OF THE INVENTION

The detailed description provided below in connection with the appended drawing is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “primer” is known by those skilled in the art, and refers to a polynucleotide that has about 10-30 nucleotides (for example, 10, 11, 12, 13, 14, 15, 16, 17, 18, 19, 20, 21, 22, 23, 24, 25, 26, 27, 28, 29 or 30; preferably 15-25 nucleotides) in length. A primer can act as a point of initiation of synthesis on a polynucleotide sequence when placed under appropriate conditions (e.g., PCR). The primer will be completely or substantially complementary to a region of the target polynucleotide sequence to be copied. Therefore, under conditions conducive to hybridization, the primer will anneal to the complementary region of the target sequence. Upon addition of suitable reactants, which include, but are not limited to, a polymerase, nucleotide triphosphates, etc., the primer is extended by the polymerizing agent to form a copy of the target sequence. As used herein, the term “forward primer” refers to a primer that anneals to the 3′ end of a first strand of the target DNA, and the term “reverse primer” refers to a primer that anneals to the 3′ end of a second strand (complimentary to the first strand) of the target DNA.

As used herein, the term “triplex forming oligonucleotide” (TFO) refers to a molecule that binds in the major groove of duplex DNA thereby forming a triplex structure. TFOs bind to the purine-rich strand of the duplex through Hoogsteen or reverse Hoogsteen hydrogen bonding. See, for example, Yue Li et al., Cell Chemical Biology, 2016, 23: 1325-1333, or Rodrigo Maldonado et al., RNA, 2018, 24:371-380, which are incorporated herein by reference.

As used herein, the term “reverse” when used in reference to a sequence means that the sequence has the same nucleotide sequence as a sequence of interest (e.g., exon sequence), in the reverse orientation with respect to the sequence of interest. For example, if a strand comprises a hypothetical sequence “5′-TAATCCGGTT-3′” (SEQ ID NO: 4), then its reverse sequence is “5′-TTGGCCTAAT-3′” (SEQ ID NO: 27). In the present disclosure, the term “substantially reverse” is used to means that there may be one or more nucleotide variations (i.e., nucleotide changes; preferably, one or two variations; more preferably, one variation) between the reverse sequences when they are aligned. For example, in the case when a strand comprises a hypothetical sequence “5′-TAATCCGGTT-3′” (SEQ ID NO: 4), then its substantially reverse sequence may be “5′-TTGGACTAAT-3′” (SEQ ID NO: 28), in which the nucleotide “C” at position 6 in the SEQ ID NO: 4 is changed to be a nucleotide “A” in the reverse sequence of SEQ ID NO: 28; or “5′-TCGGCCTAAT-3′” (SEQ ID NO: 29), in which the nucleotide “T” at position 9 in the SEQ ID NO: 4 is changed to be a nucleotide “C” in the reverse sequence of SEQ ID NO: 29.

The term “reverse complementary” as used herein has its general meaning in the art, and refers to a sequence that is complementary to the nucleotide sequence of interest (e.g., exon sequence), in the same orientation with respect to the nucleotide sequence of interest. For example, if a strand comprises a hypothetical sequence “5′-TAATCCGGTT-3′” (SEQ ID NO: 4), then its reverse complementary sequence is “5′-ATTAGGCCAA-3′” (SEQ ID NO: 5). In the present disclosure, the term “substantially reverse complementary” is used to means that there may be one or more mismatches (preferably, one or two mismatches; more preferably, one mismatch) between the reverse complementary sequences when they are aligned. The term “mismatch” refers to a site at which a nucleobase in one sequence and a nucleobase in another sequence with which it is aligned are not complementary. The nucleic acids are “perfectly complementary” to each other when they are fully complementary across their entire length. For example, in the case when a strand comprises a hypothetical sequence “5′-TAATCCGGTT-3′” (SEQ ID NO: 4), then its substantially reverse complementary sequence may be “5′-ATTCGGCCAA-3′” (SEQ ID NO: 6), in which the nucleotide “T” at position 4 is mismatched with a nucleotide “C”; or “5′-ATTAGACCAA-3′” (SEQ ID NO: 7), in which the nucleotide “C” at position 6 is mismatched with a nucleotide “A”.

As known in the art, a nucleotide is an organic molecule composed of a nucleobase, a five-carbon sugar (ribose or deoxyribose), and a phosphate group. The nucleobase is guanine (G), adenine (A), cytosine (C) and/or thymine (T) in DNA, and is guanine (G), adenine (A), cytosine (C) and/or uracil (U) in ribonucleic acid (RNA). The term “pyrimidine nucleotide” as disclosed herein refers to any nucleotide comprising a pyrimidine base as the nucleobase, examples of which include, but are not limited to, cytosine (C), thymine (T), and uracil (U) nucleotides. A pyrimidine nucleotide may be a ribose sugar molecule (i.e., a “pyrimidine ribonucleotide”) or a deoxyribose sugar molecule (i.e., a “pyrimidine deoxyribonucleotide”). The term “purine nucleotide” refers to any nucleotide comprising a purine base as the nucleobase, examples of which include, but are not limited to, adenine (A) and guanine (G) nucleotides. A purine nucleotide may be a ribose sugar molecule (i.e., a “purine ribonucleotide”) or a deoxyribose sugar molecule (i.e., a “purine deoxyribonucleotide”).

As used herein, the term “polypurine sequence” refers to a nucleotide sequence, in which at least 90% (e.g., 90%, 91%, 92%, 93%, 94%, 95%, 96%, 97%, 98%, 99% or 100%) of the nucleotides comprise a purine base (i.e., adenine or guanine) as the nucleobase. The term “polypyrimidine sequence” refers to a nucleotide sequence, in which at least 90% (e.g., 90%, 91%, 92%, 93%, 94%, 95%, 96%, 97%, 98%, 99% or 100%) of the nucleotides comprise a pyrimidine base (i.e., cytosine, thymine, or uracil nucleobase) as the nucleobase.

As known in the art, “Hoogsteen base pairing” and “reverse Hoogsteen base pairing” are intended to denote pairings which do not follow the Watson-Crick base pairing rule. Specifically, in the Hoogsteen base pairing, the distance between the C1′ and C1′ atoms is about 8.6 Å, which is approximately 2 Å shorter as compared to that in the Watson-Crick base pairing, in which the pyrimidine base uses its Watson-Crick surface to pair with the N1, C6, N7 side of the purine base, and the pyrimidine base rotates 180° with respect to the purine base thereby forming the Hoogsteen base pairing. Two strands forming the Hoogsteen base pairing are in parallel orientation. By contrast, in the reverse Hoogsteen base pairing, purine base binds with purine base (for example, G*G or A*A pairing), in which two strands forming the reverse Hoogsteen base pairing are in antiparallel orientation. For example, see, U.S. Pat. No. 6,727,059 B1, WO 2012/139628 Å1, and Atul Rangadurai, J Magn Reson., 2019, 308: 106589, which are incorporated herein by reference.

The term “fluorophore” as used herein refers to a molecule that absorbs energy of a specific wavelength (i.e., a first wavelength) and re-emits energy at a different wavelength (i.e., a second wavelength). For example, in the case when the fluorophore is FITC, it may be excited by a light having a wavelength of 485-495 nm (a first wavelength), and emits light at 520-530 nm (a second wavelength).

### II. Description of the Invention

The present disclosure is based, at least in part, on the discovery that the substitution of thymine nucleotide(s) in dsDNA with modified dUTPs could improve the binding affinity of TFO to the dsDNA. Based on this discovery, the present invention aims at providing a method of detecting a target sequence in a double-stranded nucleic acid (e.g., a dsDNA) by using TFO, as well as kits for such purpose.

Accordingly, the first aspect of the present disclosure is directed to a method of detecting a target sequence in a double-stranded nucleic acid. According to embodiments of the present disclosure, the method comprises,
(a) mixing the double-stranded nucleic acid with PCR reagents;
(b) subjecting the mixture of step (a) to PCR;
(c) mixing the amplified product of step (b) with a TFO;
(d) irradiating the triplex complex of step (c) with a light having a first wavelength; and
(e) measuring the signal emitted from the triplex complex at a second wavelength to detect the target sequence.

In step (a), the double-stranded nucleic acid is mixed with the PCR reagents, which comprise a forward primer, a reverse primer, a DNA polymerase, a reaction buffer (e.g., Tris-HCL), and dNTPs. As known in the art, the forward primer and reverse primer respectively anneal to specific sequences in the double-stranded nucleic acid, thereby amplifying a desired fragment via PCR. In general, the forward primer anneals to the 3′ end of the first strand (e.g., the antisense strand or sense strand) of the double-stranded nucleic acid, and the reverse primer anneals to the 3′ end of the second strand (complementary to the first strand; e.g., the sense strand or antisense strand) of the double-stranded nucleic acid. In an appropriate reaction (for example, PCR), the forward primer and reverse primer respectively serve as starting materials for DNA polymerization that amplifies the desired fragment (e.g., the present target sequence) by using dNTPs as building blocks with the aid of DNA polymerase. A person having ordinary skill in the art may design the primer sequences in accordance with intended purpose. The methods for designing and producing primers are known in the art; hence, the detailed description thereof is omitted herein for the sake of brevity.

According to some embodiments of the present disclosure, the forward primer is linked to a first fluorophore (e.g., a donor fluorophore or an acceptor fluorophore), and the dNTPs consist of dATP, dCTP, dGTP, and dUTP. The present method is characterized in that, (i) the PCR reagents do not comprise dTTP; accordingly, the thus-amplified sequence is composed of A, C, G and U nucleotides only, without the presence of T nucleotide; and (ii) the dUTP is a modified dUTP. Examples of the modified dUTP suitable for use in the present method include, but are not limited to,
(i) Br-dUTP, which has the structure of

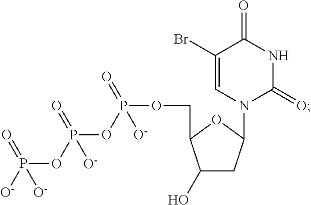 [SMILES: O=c1[nH]c(=O)n(C2CC(O)C(COP(=O)([O-])OP(=O)([O-])OP(=O)([O-])[O-])O2)cc1Br]

(ii) Cl-dUTP, which has the structure of;

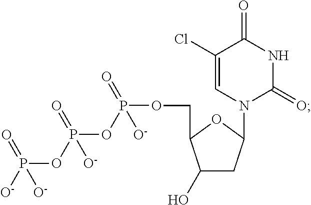 [SMILES: O=c1[nH]c(=O)n(C2CC(O)C(COP(=O)([O-])OP(=O)([O-])OP(=O)([O-])[O-])O2)cc1Cl]

(iii) F-dUTP, which has the structure of

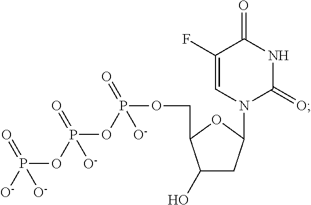 [SMILES: O=c1[nH]c(=O)n(C2CC(O)C(COP(=O)([O-])OP(=O)([O-])OP(=O)([O-])[O-])O2)cc1F]

and
(iv) Fml-dUTP, which has the structure of

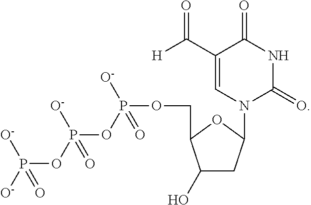 [SMILES: O=Cc1cn(C2CC(O)C(COP(=O)([O-])OP(=O)([O-])OP(=O)([O-])[O-])O2)c(=O)[nH]c1=O]

In some preferred embodiments, the dUTP is the Br-dUTP.

Preferably, the PCR reagents further comprise MgCl₂.

In step (b), the mixture of step (a) is subjected to PCR so that the target sequence is amplified. In general, a PCR protocol includes, (i) incubating the mixture at 94° C. for 3 minutes to completely denature the double-stranded nucleic acid; (ii) incubating at 94° C. for 45 seconds (denaturation step); (iii) incubating at 55-65° C. for 30 seconds (annealing step); and (iv) incubating at 72° C. for 1 minute to 1 minute and 30 seconds (extension step). The PCR amplification (i.e., the denaturation, annealing and extension steps) is performed for 25-30 cycles. As could be appreciated, a person having ordinary skill in the art may adjust the PCR condition in accordance with the general knowledge in the art and the intended purposes (for example, the sequence intended to be amplified, and/or the length of the primers).

In step (c), the amplified product of step (b) is mixed with the TFO so as to produce the triplex complex (including the first and second strands amplified in step (b), and the TFO serving as the third strand). As described above, the TFO may serve as a probe to detect specific nucleic acid sequence. The design and production of the TFO are known to any skilled artisan in the art. A persona having ordinary skill in the art may choose and/or design a suitable TFO in accordance with the general knowledge in the art and the intended purposes (for example, the sequence intended to be detected). According to certain embodiments of the present disclosure, the present TFO may bind to a DNA fragment, in which the number of T nucleotides in the DNA fragment is 0-5 (e.g., 0, 1, 2, 3, 4, or 5); preferably, 0-2 (e.g., 0, 1, or 2). According to some embodiments, the present TFO consists of 9-40 nucleotides (preferably, 10-25 nucleotides), and is substantially reverse to a strand amplified by the forward primer, i.e., the TFO is reverse to the strand of nucleic acid amplified by the forward primer, in which one or two nucleotide variations may be present between the TFO and amplified strand. In certain preferred embodiments, the sequence of the TFO partially overlaps with the sequence of the forward primer. For example, according to one exemplary embodiment of the present disclosure, the target sequence is PD-L1, which includes a first strand (“AGCAGAGGAGGAGAATGAAGAAAGA”, from 5′ end to 3′ end; SEQ ID NO: 8) and a second strand (“TCTTTCTTCATTCTCCTCCTCTGCT”, from 5′ end to 3′ end; SEQ ID NO: 9) complementary to the first strand, in which the forward primer used to amplify the second strand has the nucleotide sequence of “AGCAGAGGAGGAGAA” (from 5′ end to 3′ end; SEQ ID NO: 1), and the TFO binding to the forward primer (via reverse Hoogsteen base pairing) has the nucleotide sequence of “AGAAAGAAGGAAGAGGAGGAGA” (from 5′ end to 3′ end; SEQ ID NO: 3); in this embodiment, the TFO is reverse to the first strand, with one nucleotide variation present therebetween, i.e., reverse complementary to the second strand, with one mismatch base pair therebetween, see, the nucleotide “G” of the TFO sequence (SEQ ID NO: 3) marked in bold font, the nucleotide “T” of the first strand (SEQ ID NO: 8) marked in bold font, and the nucleotide “A” of the second strand (SEQ ID NO: 9) marked in bold font.

Preferably, no more than one (i.e., one or zero) pyrimidine nucleotide (i.e., T and C nucleotides) is present in the nucleotide sequence of the TFO. More preferably, the TFO consists of A and G nucleotides.

According to certain embodiments, the TFO is linked to an acceptor fluorophore. In the present disclosure, once the TFO binds to the amplified sequence, the energy would be transferred from the forward primer-linked donor fluorophore to the TFO-linked acceptor fluorophore, a process known as “fluorescence resonance energy transfer” (FRET). As known by a skilled artisan, FRET refers to the energy transfer between two fluorophores (i.e., the donor fluorophore and the acceptor fluorophore), in which the emission spectrum of the donor fluorophore overlaps the absorption (excitation) spectrum of the acceptor fluorophore; in this case, when two fluorophores are in close proximity (for example, less than 10 nm), energy is passed non-radiatively between the fluorophores that leads to a reduction in the donor's fluorescence intensity and an increase in the acceptor's emission intensity. FRET is a technique widely used in the art to investigate molecular interaction and protein binding. As would be appreciated, a skilled artisan may choose suitable donor and acceptor fluorophores in accordance with the general knowledge in the art and the intended purposes. Exemplary fluorophores suitable for use in the present invention include, but are not limited to, cyan fluorescent protein (CFP), green fluorescent protein (GFP), yellow fluorescent protein (CFP), red fluorescent protein (CFP), far-red fluorescent protein (FFP), infrared fluorescent protein (IFP), fluorescein isothiocyanate (FITC), phycoerythrin (PE), allophycocyanin (APC), carboxy-X-rhodamine (ROX), Texas red, cyanine dye (for example, Cy2, Cy3, Cy5 or Cy7), and Alexa Fluor© day (e.g., Alexa Fluor®488, Alexa Fluor®430, Alexa Fluor®532, Alexa Fluor®546, Alexa Fluor®555, Alexa Fluor®568, Alexa Fluor®594, Alexa Fluor®644, Alexa Fluor®660 or Alexa Fluor®680). In one exemplary embodiment, the donor fluorophore and the acceptor fluorophore are respectively FITC and ROX.

According to some embodiments of the present disclosure, the donor fluorophore (e.g. FITC) is linked to the 5′ end of the forward primer, and the acceptor fluorophore (e.g., ROX) is linked to the 3′ end of the TFO.

Optionally, the method further comprises a step of incubating the product of step (c) at a suitable condition (i.e., step (c-1)), prior to the step (d). According to some embodiments, the mixture of the TFO and PCR amplified product is incubated at 37° C. to 40° C. (such as 37° C., 37.1° C., 37.2° C., 37.3° C., 37.4° C., 37.5° C., 37.6° C., 37.7° C., 37.8° C., 37.9° C., 38° C., 38.1° C., 38.2° C., 38.3° C., 38.4° C., 38.5° C., 38.6° C., 38.7° C., 38.8° C., 38.9° C., 39° C., 39.1° C., 39.2° C., 39.3° C., 39.4° C., 39.5° C., 39.6° C., 39.7° C., 39.8° C., 39.9° C., or 40° C.) for 30-60 minutes (such as 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes), thereby forming the triplex complex. As could be appreciated, the reaction temperature and time vary with the TFO and amplified sequence. A skilled artisan may adjust the reaction temperature and/or time in accordance with practical uses. In one exemplary embodiment, the mixture of the TFO and PCR amplified product is incubated at 37° C. for 60 minutes to form the triplex complex. In another exemplary embodiment, the mixture of the TFO and PCR amplified product is incubated at 40° C. for 30 minutes to form the triplex complex.

According to certain embodiments, the mixture of step (c) (i.e., the mixture of the TFO and PCR amplified product) further comprises MgCl₂, which is present in the mixture at a concentration of 2-10 mM; for example, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 mM. In one exemplary embodiment, MgCl₂ is present in the mixture of step (c) at a concentration of 2 mM. In another exemplary embodiment, MgCl₂ is present in the mixture of step (c) at a concentration of 10 mM.

Next, the product of step (c) is irradiated with a light having a first wavelength to excite the donor fluorophore (step (d)), and then the signal emitted from the acceptor fluorophore at a second wavelength was measured to detect the target sequence (step (e)). As would be appreciated, the first and second wavelengths vary with the donor and acceptor fluorophores selected. A skilled artisan may choose suitable first and second wavelengths in accordance with the general knowledge in the art (i.e., the excitation/absorption and emission spectrums corresponding to the intended fluorophores). According to one exemplary embodiment, the donor and acceptor fluorophores are respectively FITC and ROX. In the embodiment, the first and second wavelengths are respectively 485-495 nm and 600-605 nm.

The second aspect of the present disclosure pertains to a method of determining whether a target sequence (e.g., PD-L1) is present in a biological sample. The method comprises isolating DNA from the biological sample, and then performing the detection method as described in the first aspect (i.e., the aforementioned steps (a)-(e)), in which in the case when the fluorophore is detected at the second wavelength, then the target sequence is present in the biological sample; on the contrary, in the case when no signal of the fluorophore is detected at the second wavelength, then the target sequence is absent in the biological sample. By this way, different diseases (e.g., cancers) may be diagnosed or prognosed based on the detection results.

According to certain embodiments of the present disclosure, the target sequence is derived from Staphylococcus aureus, which comprised a first strand (“AAACCCCCACTGCAATGATTATCGCAATGGGGGAAAGAGGGGACTTAAAGCATAT GTTTAGCTTTGAATACTTAAAATTCTCTTGCTATT”, from 5′ end to 3′ end; SEQ ID NO: 30) and a second strand (“AATAGCAAGAGAATTTTAAGTATTCAAAGCTAAACATATGCTTTAAGTCCCCTCTT TCCCCCATTGCGATAATCATTGCAGTGGGGGTTT”, from 5′ end to 3′ end; SEQ ID NO: 31) complementary to the first strand. In these embodiments, the presence of the target sequence indicated that the subject from which the biological sample is derived/isolated is infected by Staphylococcus aureus.

The third aspect of the present disclosure provides a kit for detecting a target sequence in a double-stranded nucleic acid (e.g., a DNA). The present kit comprises, (1) a forward primer and a reverse primer corresponding to the target sequence, in which the forward primer is linked to a first fluorophore (e.g., a donor fluorophore; for example, FITC); (2) a TFO consisting of 9-40 nucleotides (preferably, 10-25 nucleotides) that is substantially reverse to a strand amplified by the forward primer, and is linked to a second fluorophore (e.g., an acceptor fluorophore; for example, ROX); and (3) dNTPs consisting of a dATP, a dCTP, a dGTP and a modified dUTP (for example, Br-dUTP, Cl-dUTP, F-dUTP or Fml-dUTP).

The present kit is characterized by not comprising a dTTP.

The forward primer, reverse primer, TFO and dNTPs of the present kit are similar to that of the method as described in the first aspect of the present disclosure. Thus, the detailed description thereof is omitted herein for the sake of brevity.

Optionally, the present kit further comprises a DNA polymerase, a reaction buffer (e.g., Tris-HCL) and MgCl₂.

According to one exemplary embodiment, the target sequence is PD-L1. In the embodiment, the forward and reverse primers of the present kit respectively comprise the nucleotide sequences of “AGCAGAGGAGGAGAA” (SEQ ID NO: 1) and “TTGTTCAGAAGTATCCTTTC” (SEQ ID NO: 2), and the TFO comprises the nucleotide sequence of “AGAAAGAAGGAAGAGGAGGAGA” (SEQ ID NO: 3).

According to another exemplary embodiment, the target sequence is derived from Staphylococcus aureus, and comprises a first strand of SEQ ID NO: 30 and a second strand of SEQ ID NO: 31. In the embodiment, the TFO used to detect the target sequence comprises the nucleotide sequence of “AGGGGAGAAAGGGGGTAA” (SEQ ID NO: 32) or “GGGAGAAAGGGGGTAA” (SEQ ID NO: 33).

Also disclosed herein is a triple-stranded nucleic acid comprising a double-stranded nucleic acid (e.g., a DNA) and a TFO, in which the double-stranded nucleic acid comprises a first strand (e.g., the sense strand or antisense strand of DNA), and a second strand complementary to the first strand (e.g., the antisense strand or sense strand of DNA). According to some embodiments of the present disclosure, the first strand of the double-stranded nucleic acid is a polypurine sequence (preferably, one or less than one pyrimidine nucleotide is present in the polypurine sequence), and the second strand of the double-stranded nucleic acid is a polypyrimidine sequence, wherein the first strand binds to the TFO via Hoogsteen base pairing or reverse Hoogsteen base pairing, and the second strand comprises a plurality of (such as 1, 2, 3, 4, 5, 6, 7, 8, 9, 10 or more) modified nucleotides. In the embodiments, the modified nucleotide is a modified U nucleotide selected from the group consisting of Br-dUTP, Cl-dUTP, F-dUTP, and Fml-dUTP. In one preferred embodiment, the modified nucleotide is the Br-dUTP.

Preferably, the second strand consists of C, A, G and modified U nucleotides, without including T nucleotide.

In various embodiments, the double-stranded nucleic acid is produced by a reaction catalyzed by a polymerase (e.g., a DNA polymerase), i.e., a polymerase-catalyzed reaction for amplifying a template nucleic acid. Exemplary reactions catalyzed by a polymerase include, but are not limited to, PCR, LAMP, RPA, etc.

Preferably, the double-stranded nucleic acid consists of 9-40 base pairs (i.e., the first and second strand respectively have 9-40 nucleotides in length); for example, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs. As would be appreciated, the lengths of the TFO and double-stranded nucleic acid may be the same or different. Preferably, the length of the TFO and double-stranded nucleic acid is the same. For example, in the case when the first and second strands of the double-stranded nucleic acid respectively have 9 nucleotides in length, then the TFO also has 9 nucleotides in length. Alternatively, in the case when the first and second strands of the double-stranded nucleic acid respectively have 22 nucleotides in length, then the TFO also has 22 nucleotides in length.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Synthesis of DNA Fragments

Five dsDNAs (respectively designated as “double-stranded fragment 1”, “double-stranded fragment 2”, “double-stranded fragment 2-1”, “double-stranded fragment 2-2” and “double-stranded fragment 3”), four TFO probes (respectively designated as “G-TFO”, “A-TFO”, “C-TFO” and “T-TFO”), and three G-TFO probe fragments (respectively designated as “G-TFO-20”, “G-TFO-17” and “G-TFO-14”) were synthesized in the study. The nucleotide sequences of the synthesized fragments were summarized in Table 1.

TABLE 1
Nucleotide sequences of the present synthetic fragments

| Name | Nucleotide sequence from 5′ end | |
|---|---|---|
| (base pair) | to 3′ end (SEQ ID NO) | Note |
| double-stranded | GAATTCCAAGCTTCATGCTACGTTGG | The sequence binding to G-TFO as |
| fragment 1_ | AGAAGGAGGAGAAGGAAAGAGTCC | marked in bold font consisted of |
| positive strand | TCTATACGAGATCTGGATC | purine nucleotides |
| (69 bp) | (SEQ ID NO: 10) | |
| double-stranded | GATCCAGATCTCGTATAGAGGACTCT | Complementary to the double- |
| fragment 1_ | TTCCTTCTCCTCCTTCTCCAACGTA | stranded fragment 1_positive |
| negative strand | GCATGAAGCTTGGAATTC | strand, in which the sequence |
| (69 bp) | (SEQ ID NO: 11) | corresponding to the G-TFO |
| | | binding sequence of the double- |
| | | stranded fragment 1_positive strand |
| | | was marked in bold font |
| double-stranded | GAATTCCAAGCTTCATGCTACGTTGG | The sequence binding to G-TFO as |
| fragment 2_ | AGAAGTAGGAGAAGGAAAGAGTCC | marked in bold font had one T |
| positive strand | TCTATACGAGATCTGGATC | nucleotide (underlined) |
| (69 bp) | (SEQ ID NO: 12) | |
| double-stranded | GATCCAGATCTCGTATAGAGGACTCT | Complementary to the double- |
| fragment 2_ | TTCCTTCTCCTACTTCTCCAACGTA | stranded fragment 2_positive |
| negative strand | GCATGAAGCTTGGAATTC | strand, in which the sequence |
| (69 bp) | (SEQ ID NO: 13) | corresponding to the G-TFO |
| | | binding sequence of the double- |
| | | stranded fragment 2_positive strand |
| | | was marked in bold font |
| double-stranded | GAATTCCAAGCTTCATGCTACGTTGG | The sequence binding to G-TFO as |
| fragment 2- | AGAAGTTGGAGAAGGAAAGAGTCC | marked in bold font had two T |
| 1_positive strand | TCTATACGAGATCTGGATC | nucleotides (underlined) |
| (69 bp) | (SEQ ID NO: 14) | |
| double-stranded | GATCCAGATCTCGTATAGAGGACTCT | Complementary to the double- |
| fragment 2- | TTCCTTCTCCAACTTCTCCAACGTA | stranded fragment 2-1_positive |
| 1_negative strand | GCATGAAGCTTGGAATTC | strand, in which the sequence |
| (69 bp) | (SEQ ID NO: 15) | corresponding to the G-TFO |
| | | binding sequence of the double- |
| | | stranded fragment 2-1_positive |
| | | strand was marked in bold font |
| double-stranded | GAATTCCAAGCTTCATGCTACGTTGG | The sequence binding to G-TFO as |
| fragment 2- | AGAAGTAGGAGAAGTAAAGAGTCC | marked in bold font had two T |
| 2_positive strand | TCTATACGAGATCTGGATC | nucleotides (underlined) |
| (69 bp) | (SEQ ID NO: 16) | |
| double-stranded | GATCCAGATCTCGTATAGAGGACTCT | Complementary to the double- |
| fragment 2- | TTACTTCTCCTACTTCTCCAACGTA | stranded fragment 2-2_positive |
| 2_negative strand | GCATGAAGCTTGGAATTC | strand, in which the sequence |
| (69 bp) | (SEQ ID NO: 17) | corresponding to the G-TFO |
| | | binding sequence of the double- |
| | | stranded fragment 2-2_positive |
| | | strand was marked in bold font |
| double-stranded | GAATTCCAAGCTTCATGCTACGTTGG | The sequence binding to G-TFO as |
| fragment | AGAAGCAGGAGAAGGAAAGAGTCC | marked in bold font had one C |
| 3_positive strand | TCTATACGAGATCTGGATC | nucleotide (underlined) |
| (69 bp) | (SEQ ID NO: 18) | |
| double-stranded | GATCCAGATCTCGTATAGAGGACTCT | Complementary to the double- |
| fragment | TTCCTTCTCCTGCTTCTCCAACGTA | stranded fragment 3_positive |
| 3_negative strand | GCATGAAGCTTGGAATTC | strand, in which the sequence |
| (69 bp) | (SEQ ID NO: 19) | corresponding to the G-TFO |
| | | binding sequence of the double- |
| | | stranded fragment 3_positive strand |
| | | was marked in bold font |
| G-TFO | GAGAAAGGAAGAGGAGGAAGAGG | Reverse to the marked sequence of |
| (23 bp) | (SEQ ID NO: 20) | double-stranded fragment |
| | | 1_positive strand |
| A-TFO | GAGAAAGGAAGAGGAAGAAGAGG | Substantially reverse to the marked |
| (23 bp) | (SEQ ID NO: 21) | sequence of double-stranded |
| | | fragment 1_positive strand, and |
| | | having one A variation (underlined) |
| C-TFO | GAGAAAGGAAGAGGACGAAGAGG | Substantially reverse to the marked |
| (23 bp) | (SEQ ID NO: 22) | sequence of double-stranded |
| | | fragment 1_positive strand, and |
| | | having one C variation (underlined) |
| T-TFO | GAGAAAGGAAGAGGATGAAGAGG | Substantially reverse to the marked |
| (23 bp) | (SEQ ID NO: 23) | sequence of double-stranded |
| | | fragment 1_positive strand, and |
| | | having one T variation (underlined) |
| G-TFO-20 | AAAGGAAGAGGAGGAAGAGG | G-TFO fragment lacking the first |
| (20 bp) | (SEQ ID NO: 24) | three nucleotides of G-TFO |
| G-TFO-17 | GGAAGAGGAGGAAGAGG | G-TFO fragment lacking the first |
| (17 bp) | (SEQ ID NO: 25) | six nucleotides of G-TFO |
| G-TFO-14 | AGAGGAGGAAGAGG | G-TFO fragment lacking the first |
| (14 bp) | (SEQ ID NO: 26) | nine nucleotides of G-TFO |

In addition, a dsDNA (designated as “SAT-1”) derived from the genome of Staphylococcus aureus and four TFO probe fragments (respectively designated as “SAT-1-p18”, “SAT-1-p16”, “SAT-1-p14” and “SAT-1-p12”) for detecting the SAT-1 dsDNA were synthesized in the study. The nucleotide sequences of the synthesized fragments were summarized in Table 2.

TABLE 2
Nucleotide sequences of the present synthetic fragments

| Name | Nucleotide sequence from 5′ | |
|---|---|---|
| (base pair) | end to 3′ end (SEQ ID NO) | Note |
| SAT-1_ | AAACCCCCACTGCAATGATTATCGCA | The sequence binding to TFO as |
| positive strand | ATGGGGGAAAGAGGGGACTTAAAG | marked in bold font had one T |
| (90 bp) | CATATGTTTAGCTTTGAATACTTAAAA | nucleotide (underlined) |
| | TTCTCTTGCTATT | |
| | (SEQ ID NO: 30) | |
| SAT-1_ | AATAGCAAGAGAATTTTAAGTATTCA | Complementary to the SAT- |
| negative strand | AAGCTAAACATATGCTTTAAGTCCCC | 1_positive strand, in which the |
| (90 bp) | TCTTTCCCCCATTGCGATAATCATTG | sequence corresponding to the TFO |
| | CAGTGGGGGTTT | binding sequence of the SAT- |
| | (SEQ ID NO: 31) | 1_positive strand was marked in |
| | | bold font |
| SAT-1-p18 | AGGGGAGAAAGGGGGTAA | Reverse to the marked sequence of |
| (18 bp) | (SEQ ID NO: 32) | SAT-1_positive strand |
| SAT-1-p16 | GGGAGAAAGGGGGTAA | TFO fragment lacking the first two |
| (16 bp) | (SEQ ID NO: 33) | nucleotides of TFO SAT-1-p18 |
| SAT-1-p14 | GAGAAAGGGGGTAA | TFO fragment lacking the first four |
| (14 bp) | (SEQ ID NO: 34) | nucleotides of TFO SAT-1-p18 |
| SAT-1-p12 | GAAAGGGGGTAA | TFO fragment lacking the first six |
| (12 bp) | (SEQ ID NO: 35) | nucleotides of TFO SAT-1-p18 |

Preparation of Modified dsDNAs

The modified dsDNAs were produced by PCR technology. In the process of synthesizing dsDNA by PCR using specific primers, the dNTPs of PCR reagents consisted of dATP, dTTP, dCTP and dGTP. By contrast, in the process of synthesizing modified dsDNA, the dTTP or dCTP of the dNTPs was replaced by a modified dUTP or a modified dCTP.

Specifically, for the purpose of producing the double-stranded fragment 1 or double-stranded fragment 2 containing modified dUTP, the dTTP of the dNTPs was replaced by Br-dUTP, Cl-dUTP, F-dUTP or Fml-dUTP. The thus-produced polynucleotide consisted of A, G, C and modified U (i.e., Br-U, Cl-U, F-U or Fml-U) nucleotides, without including T nucleotide. According to the modified group, the modified double-stranded fragment 1 was designated as “double-stranded fragment 1-Br-U”, “double-stranded fragment 1-Cl-U”, “double-stranded fragment 1-F-U”, or “double-stranded fragment 1-Fml-U”; and the modified double-stranded fragment 2 was designated as “double-stranded fragment 2-Br-U”, “double-stranded fragment 2-Cl-U”, “double-stranded fragment 2-F-U”, or “double-stranded fragment 2-Fml-U”.

For the purpose of producing the double-stranded fragment 3 containing modified dCTP, the dCTP of the dNTPs was replaced by 5-hydroxy-dCTP (hereinafter as “OH-dCTP”), 5-bromo-dCTP (hereinafter as “Br-dCTP”), or 5-aminoallyl-dCTP (hereinafter as “AA-dCTP”). The thus-produced polynucleotide consisted of A, G, T and modified C (i.e., OH-C, Br-C or AA-C) nucleotides. According to the modified group, the modified double-stranded fragment 3 was designated as “double-stranded fragment 3-OH-C”, “double-stranded fragment 3-Br-C”, or “double-stranded fragment 3-AA-C”.

On the other hand, the SAT-1 dsDNA containing modified dUTP was produced by PCR via replacing the dTTP of the dNTPs with Br-dUTP. The thus-produced modified SAT-1 dsDNA designated as “SAT-1-Br-U” consisted of A, G, C and modified U (i.e., Br-U) nucleotides, without including T nucleotide.

### Formation of Triple Helix (Triplex Complex)

0.5 M dsDNA template was mixed with 2 M TFO in 10 mM Tris-HCl (pH=7.2) containing 2 mM MgCl₂, followed by incubating at 37° C. for 60 minutes or at 40° C. for 30 minutes. The formation of triple helix was determined by polyacrylamide gel electrophoresis (PAGE).

### FRET Detecting System

In the present study, the DNA fragment binding to the TFO probe preferably had only 1 or 2 pyrimidine nucleotide(s) in its nucleotide sequence. The PD-L1 gene served as an example for testing the use of the TFO probe and PD-L1 primers in detecting a target sequence in the PD-L1 gene. To this purpose, the sequence of the PD-L1 gene targeted by the TFO probe (i.e., the TFO-binding sequence) was first determined, in which only one T nucleotide was present in the TFO-binding sequence. The forward primer was located upstream of the TFO-binding site, and had a FITC fluorophore linked to its 5′ end. The TFO probe had a ROX fluorophore linked to its 3′ end.

The mixture of the primer pair (SEQ ID NOs: 1 and 2, in which the FITC fluorophore was linked to the 5′ end of the forward primer), non-modified or Br-U-modified PD-L1 gene fragment (i.e., PD-L1 Dx or PD-L1 Dx-Br-U), and TFO probe (PD-L1-TFO; SEQ ID NO: 3, in which the ROX fluorophore was linked to the 3′ end of the PD-L1-TFO) was incubated at 40° C. for 30 minutes, followed by irradiating with a light having a wavelength of 488 nm to excite the FITC fluorophore, and then detecting the signal emitted from the ROX fluorophore at the wavelength of 605 nm. In the present method, the detection of ROX signal indicated the presence of the triplex complex; on the contrary, no detection of ROX signal indicated the absence of the triplex complex.

### Example 1 Effect of T Nucleotide in DNA Sequence on TFO Binding

Double-stranded fragment 1 and double-stranded fragment 2 were used to determine whether the presence of T nucleotide in DNA sequence would affect the binding affinity of TFO to the DNA sequence, in which the double-stranded fragment 1 was perfectly complementary to the G-TFO (the binding sequence of the positive strand of the double-stranded fragment 1 was fully complementary to the G-TFO via reverse Hoogsteen base pairing), and the double-stranded fragment 2 included one nucleotide substitution in the center of the G-TFO binding sequence (the G nucleotide was substituted by a T nucleotide; see, Table 1, the underlined T nucleotide of SEQ ID NO: 12).

The data indicated that in the reaction containing 10 mM MgCl₂, the double-stranded fragment 1 bound to the G-TFO and formed triplex complexes, while only 50% of the double-stranded fragment 2 exhibited binding affinity to the G-TFO and formed triplex complexes (data not shown). The data demonstrated that the presence of T nucleotide (even only one T nucleotide) in the TFO-binding sequence would greatly affect the formation of triplex complexes.

Next, the concentration of MgCl₂ in PCR reagents was reduced from 10 mM to 5 mM, 2 mM, or 1 mM. The results indicated that the percentage of triplex complexes formed by the G-TFO and the double-stranded fragment 1 or double-stranded fragment 2 decreased with the decrease of MgCl₂ concentration (data not shown); suggesting that the formation of triplex complexes by TFO and dsDNA was proportional to the concentration of MgCl₂.

According to the analytic results, 2 mM MgCl₂ was used to carry out the following experiments evaluating the binding affinity of TFO to modified dsDNAs, due to that MgCl₂ at a concentration of 2 mM allowed the TFO to form triplex complexes with the double-stranded fragment 1, without interacting with the double-stranded fragment 2 forming triplex complexes.

### Example 2 Binding Affinity of TFO to Modified dsDNAs

### 2.1 Binding Affinity of TFO to Double-Stranded Fragment 2-Br-U, Double-Stranded Fragment 2-F-U or Double-Stranded Fragment 2-Fml-U

In this example, the double-stranded fragment 1, double-stranded fragment 2 and modified double-stranded fragment 2 (including double-stranded fragment 2-Br-U, double-stranded fragment 2-F-U and double-stranded fragment 2-Fml-U) were respectively mixed with different types of TFOs (including G-TFO, A-TFO, C-TFO, and T-TFO), and then incubated at 37° C. for 1 hour. The formation of triplex complexes was determined by PAGE.

In the reaction containing 2 mM MgCl₂, the double-stranded fragment 1 exhibited a high binding affinity to the G-TFO and a low binding affinity to the A-TFO (data not shown). Compared to the non-modified double-stranded fragment 2, which did not bind to the tested TFOs (including the G-TFO, A-TFO, C-TFO and T-TFO), the modified double-stranded fragment 2 (i.e., double-stranded fragment 2-Br-U, double-stranded fragment 2-F-U and double-stranded fragment 2-Fml-U) bound to the tested TFOs and formed triplex complexes therewith, in which the modified double-stranded fragment 2 exhibited a high binding affinity to the G-TFO, A-TFO and C-TFO, and a low binding affinity to the T-TFO (data not shown). The data indicated that the substitution of T nucleotide in the TFO-binding sequence of dsDNA with a modified U nucleotide improved the binding affinity of the dsDNA to TFO. Surprisingly, the binding affinity of the modified double-stranded fragment 2 (including double-stranded fragment 2-Br-U, double-stranded fragment 2-F-U and double-stranded fragment 2-Fml-U) to the TFO was greater than that of the double-stranded fragment 1 to the TFO (data not shown).

After confirming the substitution of T nucleotide of the double-stranded fragment 1 or double-stranded fragment 2 with a Br-U, F-U or Fml-U nucleotide improved the binding affinity of the double-stranded fragment 1 or double-stranded fragment 2 to the TFO, the nucleotide modification exhibiting the best improvement was then determined by dissociation constant (K_{d}). According to the results of Table 3, the G-TFO exhibited binding affinity towards double-stranded fragment 1 with or without nucleotide modification, including non-modified double-stranded fragment 1 (K_{d}=8.96×10⁸ M), double-stranded fragment 1-F-U (K_{d}=2.10×10⁻⁸ M), double-stranded fragment 1-Br-U (K_{d}=6.17×10⁸ M), and double-stranded fragment 1-Fml-U (K_{d}=6.55×10⁻⁸ M). The data of Table 3 further indicated that the K_{d} value of the interaction between the double-stranded fragment 2-Br-U and G-TFO was about 6.52×10⁻⁸ M; the K_{d} value of the interaction between the double-stranded fragment 2-F-U and G-TFO was about 1.04×10⁻⁷ M; and the K_{d} value of the interaction between the double-stranded fragment 2-Fml-U and G-TFO was about 7.28×10⁻⁸ M, while no binding affinity was detected between non-modified double-stranded fragment 2 and G-FTO. The data demonstrated that in the tested nucleotide substitutions, the double-stranded fragment 2-Br-U exhibited the highest binding affinity to the G-TFO. Accordingly, the T nucleotide in the TFO-binding sequence was substituted by the Br-U nucleotide in the following studies.

TABLE 3
Binding affinity of G-TFO towards
specified double-stranded fragment

| TFO | Double-stranded fragment | K_{d} value |
|---|---|---|
| G-TFO | Double-stranded fragment 1 | 8.96*10⁻⁸M |
| | Double-stranded fragment 1-F-U | 2.10*10⁻⁸M |
| | Double-stranded fragment 1-Br-U | 6.17*10⁻⁸M |
| | Double-stranded fragment 1-Fml-U | 6.55*10⁻⁸M |
| | Double-stranded fragment 2 | Undetectable |
| | Double-stranded fragment 2-F-U | 1.04*10⁻⁷M |
| | Double-stranded fragment 2-Br-U | 6.52*10⁻⁸M |
| | Double-stranded fragment 2-Fml-U | 7.28*10⁻⁸M |

### 2.2 Binding Affinity of TFO to Double-Stranded Fragment 3-OH-C, Double-Stranded Fragment 3-Br-C or Double-Stranded Fragment 3-AA-C

Compared to the double-stranded fragment 1, which is perfectly complementary to the G-TFO, the double-stranded fragment 3 included one G→C substitution in the G-TFO-binding sequence (see, Table 1, the underlined C nucleotide of SEQ ID NO: 18). According to the result of PAGE, in the reaction containing 2 mM MgCl₂, the double-stranded fragment 3 did not form triplex complexes with the tested TFO (i.e., the G-TFO, A-TFO, C-TFO and T-TFO). In the reaction containing 10 mM MgCl₂, the double-stranded fragment 3 bound to the T-TFO, G-TFO and A-TFO (binding affinity: T-TFO>G-TFO>A-TFO), but still not interacting with the C-TFO (data not shown).

Then, in the reaction containing 10 mM MgCl₂, the binding affinity of the double-stranded fragment 3-OH-C, double-stranded fragment 3-Br-C or double-stranded fragment 3-AA-C to the four TFOs was tested. The data of PAGE indicated that the double-stranded fragment 3-OH-C did not form triplex complexes with the four tested TFOs; the double-stranded fragment 3-Br-C only formed triplex complexes with the T-TFO; and the double-stranded fragment 3-AA-C formed triplex complexes with the T-TFO or G-TFO. However, the binding affinity of the double-stranded fragment 3-Br-C or double-stranded fragment 3-AA-C to the TFO was less than that of the double-stranded fragment 3 to the TFO (data not shown). The results indicated that the substitution of C nucleotide of the double-stranded fragment 3 with an OH-C, Br-C or AA-C nucleotide would reduce the binding affinity of the double-stranded fragment 3 to the TFO.

2.3 Effect of Br-U Substitution on Binding Affinity of dsDNA to TFO

After confirming the substitution of T nucleotide in TFO-binding sequence with a Br-U nucleotide improved the binding affinity of dsDNA to TFO, the dTTP of the dNTPs was replaced by a Br-dUTP in the PCR for synthesizing the double-stranded fragment 1, double-stranded fragment 2 and double-stranded fragment 3. The thus-produced modified fragments were respectively designated as “double-stranded fragment 1-Br-U”, “double-stranded fragment 2-Br-U”, and “double-stranded fragment 3-Br-U”.

The data indicated that in the reaction containing 2 mM MgCl₂, each of the double-stranded fragment 1-Br-U, double-stranded fragment 2-Br-U and double-stranded fragment 3-Br-U was capable of binding to the TFOs (including the G-TFO, A-TFO, C-TFO and T-TFO) and forming triplex complexes therewith; suggesting that the substitution of T nucleotide with BrU nucleotide not only improved the binding affinity of the TFO-binding sequence to the TFO, but also improved the capability of the whole dsDNA to form triplex complexes with the TFO.

### Example 3 Substitution of Two T Nucleotides in TFO-Binding Sequence with Br-U Nucleotides

Compared to the double-stranded fragment 1, which was perfectly complementary to the G-TFO, the double-stranded fragment 2-1 included two consecutive T nucleotides in the G-TFO-binding sequence (see, Table 1, the underlined T nucleotides of SEQ ID NO: 14). The data of PAGE indicated that in the reaction containing 2 mM MgCl₂, neither the double-stranded fragment 2-1 nor the double-stranded fragment 2-1-Br-U bound to the tested TFOs (including the A-TFO, C-TFO, G-TFO and T-TFO; data not shown). The concentration of MgCl₂ in PCR reagents was thus increased from 2 mM to 10 mM. According to the results, the double-stranded fragment 2-1 still failed to interact with the tested TFOs, while the double-stranded fragment 2-1-Br-U exhibited a high binding affinity to the A-TFO, C-TFO and G-TFO, and a low binding affinity to the T-TFO (data not shown). The data demonstrated that the substitution of two consecutive T nucleotides in the TFO-binding sequence with Br-U nucleotides was able to improve the binding affinity of the dsDNA to TFO.

Compared to the double-stranded fragment 1, the double-stranded fragment 2-2 included two non-consecutive T nucleotides in the G-TFO-binding sequence (see, Table 1, the underlined T nucleotides of SEQ ID NO: 16). The data of PAGE indicated that in the reaction containing 2 mM MgCl₂, the double-stranded fragment 2-2 cannot bind to the G-TFO, while the double-stranded fragment 2-2-Br-U exhibited a binding affinity to the G-TFO (data not shown). The results suggested that the substitution of two non-consecutive T nucleotides in the TFO-binding sequence with Br-U nucleotides was able to improve the binding affinity of the dsDNA to TFO.

### Example 4 Effect of TFO Length on dsDNA Binding

In the example, the double-stranded fragment 1, double-stranded fragment 2, double-stranded fragment 1-Br-U and double-stranded fragment 2-Br-U were respectively mixed with TFOs with different lengths (including G-TFO, G-TFO-20, G-TFO-17 and G-TFO-14), and then incubated at 37° C. for 1 hour. The formation of triplex complexes was determined by PAGE.

According to the results of PAGE, the shortest length of the TFO binding to the double-stranded fragment 1 was 17 nucleotides, and the shortest length of the TFO binding to the double-stranded fragment 1-Br-U was 14 nucleotides (data not shown). The non-modified double-stranded fragment 2 had a low binding affinity to the G-TFO-20, while the double-stranded fragment 2-Br-U exhibited a high binding affinity to the G-TFO-17 (data not shown). The data indicated that the substitution of T nucleotides with BrU nucleotides improved the binding capability of dsDNA to TFO, and accordingly shorter length of TFO was required for the formation of stable complexes with the dsDNA. The results suggested that the dsDNA with BrU modification may be detected by a short-length TFO probe.

Further, the binding affinities of SAT-1 TFO probe fragments (i.e., SAT-1-p18, SAT-1-p16, SAT-1-p14, and SAT-1-p12) towards SAT-1-Br-U were determined. According to the results, both SAT-1-p18 and SAT-1-p16 exhibited binding affinity towards SAT-1-Br-U and thus formed triple-stranded nucleic acid therewith (data not shown).

### Example 5 Detection of PD-L1 Gene

In the example, non-modified or Br-U-modified PD-L1 gene fragment (i.e., PD-L1 Dx or PD-L1 Dx-Br-U) was used to test the use of PD-L1-TFO and PD-L1 primers in detecting a target sequence in the PD-L1 gene. According to the results, in the reaction containing 10 mM MgCl₂, there was no triplex complex formed by PD-L1 Dx and PD-L1-TFO; by contrast, the PD-L1 Dx-Br-U was capable of binding to the PD-L1-TFO, and forming triplex complexes therewith (data not shown).

Then, real-time PCR was used to detect the fluorescent signal emitted from the fluorophore. As described in “Materials and Methods”, the PD-L1 Dx or PD-L1 Dx-Br-U was incubated with the PD-L1-TFO at 40° C. for 30 minutes, and the fluorescent signal was detected. As depicted in the sole FIGURE, the PD-L1 Dx-Br-U yield higher RFU values as compared to the PD-L1 Dx, suggesting the high binding affinity of the PD-L1 Dx-Br-U to PD-L1-TFO. The data confirmed that the substitution of T nucleotides in dsDNA with Br-U nucleotide improved the binding affinity of dsDNA to TFO, and the Br-U modification provides a potential means to detect target sequences in biological samples.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A triple-stranded nucleic acid comprising a double-stranded nucleic acid and a triplex forming oligonucleotide (TFO), wherein the double-stranded nucleic acid comprises a first strand and a second strand complementary to the first strand, wherein
the first strand is a polypurine sequence, which binds to the TFO via Hoogsteen base pairing or reverse Hoogsteen base pairing; and
the second strand is a polypyrimidine sequence that comprises a plurality of modified nucleotides independently selected from the group consisting of 5-fluoro-uridine, 5-chloro-uridine, 5-bromo-uridine, and 5-formyl-uridine nucleotides.

2. The triple-stranded nucleic acid of claim 1, wherein the double-stranded nucleic acid is produced by a reaction catalyzed by a polymerase.

3. The triple-stranded nucleic acid of claim 2, wherein the double-stranded nucleic acid is produced by polymerase chain reaction (PCR), loop-mediated isothermal amplification (LAMP), or recombinase polymerase amplification (RPA).

4. The triple-stranded nucleic acid of claim 1, wherein the double-stranded nucleic acid consists of 9-40 base pairs.

5. The triple-stranded nucleic acid of claim 1, wherein one or less than one pyrimidine nucleotide is present in the polypurine sequence.

6. A method of detecting a target sequence in a double-stranded nucleic acid, comprising
(a) mixing the double-stranded nucleic acid with polymerase chain reaction (PCR) reagents that comprise a forward primer, a reverse primer, a DNA polymerase, a reaction buffer, and deoxynucleotide triphosphates (dNTPs), wherein
the forward primer is linked to a first fluorophore;
the dNTPs consist of a deoxyadenosine triphosphate (dATP), a deoxycytidine triphosphate (dCTP), a deoxyguanosine triphosphate (dGTP), and a modified deoxyuridine triphosphate (dUTP) selected from the group consisting of 5-bromo-dUTP, 5-chloro-dUTP, 5-fluoro-dUTP and 5-formyl-dUTP; and
the PCR reagents do not comprise a deoxythymidine triphosphate (dTTP);
(b) subjecting the mixture of step (a) to PCR;
(c) mixing the amplified product of step (b) with a triplex forming oligonucleotide (TFO) thereby forming a triplex complex, wherein the TFO is linked to a second fluorophore, wherein one of the first and second fluorophores is a donor fluorophore, and the other of the first and second fluorophores is an acceptor fluorophore;
(d) irradiating the product of step (c) with a light having a first wavelength to excite the donor fluorophore; and
(e) measuring the signal emitted from the acceptor fluorophore at a second wavelength to detect the target sequence.

7. The method of claim 6, wherein the modified dUTP is the 5-bromo-dUTP.

8. The method of claim 6, wherein the donor fluorophore and acceptor fluorophore are respectively fluorescein isothiocyanate (FITC) and carboxy-X-rhodamine (ROX), and the first and second wavelengths are respectively 485-495 nm and 600-605 nm.

9. The method of claim 6, wherein the TFO has 9-40 nucleotides in length, and the nucleotide sequence of the TFO is substantially reverse to a strand amplified by the forward primer.

10. The method of claim 9, wherein the first fluorophore is linked to the 5′ end of the forward primer, and the second fluorophore is linked to the 3′ end of the TFO.

11. A kit for detecting a target sequence in a double-stranded nucleic acid, comprising
a forward primer and a reverse primer configured to amplify the target sequence in a polymerase chain reaction (PCR), wherein the forward primer is linked to a first fluorophore;
a triplex forming oligonucleotide (TFO) having 9-40 nucleotides in length, wherein the nucleotide sequence of the TFO is substantially reverse to a strand amplified by the forward primer, wherein the TFO is linked to a second fluorophore, wherein one of the first and second fluorophores is a donor fluorophore, and the other of the first and second fluorophores is an acceptor fluorophore; and
deoxynucleotide triphosphates (dNTPs) consisting of a deoxyadenosine triphosphate (dATP), a deoxycytidine triphosphate (dCTP), a deoxyguanosine triphosphate (dGTP), and a modified deoxyuridine triphosphate (dUTP) selected from the group consisting of 5-bromo-dUTP, 5-chloro-dUTP, 5-fluoro-dUTP and 5-formyl-dUTP;
wherein the kit does not comprise a deoxythymidine triphosphate (dTTP).

12. The kit of claim 11, further comprising a DNA polymerase, reaction buffer, and MgCl₂.

13. The kit of claim 11, wherein the first fluorophore is linked to the 5′ end of the forward primer, and the second fluorophore is linked to the 3′ end of the TFO.

14. The kit of claim 11, wherein one or less than one pyrimidine nucleotide is present in the nucleotide sequence of the TFO.

15. The kit of claim 14, wherein the TFO consists of adenine and guanine nucleotides.

16. The kit of claim 15, wherein
the target sequence is programmed death-ligand 1 (PD-L1);
the forward and reverse primers respectively comprise the nucleotide sequences of “AGCAGAGGAGGAGAA” (SEQ ID NO: 1) and “TTGTTCAGAAGTATCCTTTC” (SEQ ID NO: 2); and
the TFO comprises the nucleotide sequence of “AGAAAGAAGGAAGAGGAGGAGA” (SEQ ID NO: 3).

17. The kit of claim 11, wherein the modified dUTP is the 5-bromo-dUTP.
